(19) United States
(12) Patent Application Publication
(10) Pub. No.: US 2016/0151286 A1
(43) Pub. Date: Jun. 2, 2016

(54) Hydrophilic Microparticles, Drug-Delivery Material, Method For Manufacturing Thereof And Methods For Delivery of A Drug-Delivery Composition

(71) Applicant: TheraKine BioDelivery GmbH, Berlin (DE)

(72) Inventors: Andreas Voigt, Berlin (DE); Scott Hampton, Tarpon Springs, FL (US); Andreas Reiff, San Marino, CA (US); Sonja Lehmann, Berlin (DE)

(21) Appl. No.: 14/392,115

(22) PCT Filed: May 28, 2014
(86) PCT No.: PCT/EP2014/061184

§ 371 Date: Nov. 28, 2015

Related U.S. Application Data
(60) Provisional application No. 61/828,255, filed on May 29, 2013.

(51) Int. Cl. A61K 9/14; A61K 47/36; A61K 47/42

(52) U.S. Cl. CPC A61K 9/146; A61K 47/42; A61K 47/36

(57) ABSTRACT

Embodiments described herein relate to hydrophilic matrix material and sustained drug-delivery material, and their use in medical and cosmetic applications.

### FIELD OF THE INVENTION

Embodiments described herein relate to hydrophilic matrix material and sustained drug-delivery material, and their use in medical and cosmetic applications.

### BACKGROUND OF THE INVENTION

Pharmaceutical dosage forms include mixtures of one or more active pharmaceutical ingredients (APIs) with additional components referred to as excipients. After administration to the human or animal body, the API is intended to be released from the dosage form in order to provide the desired pharmacologic effect.

Sustained release dosage forms for delivering APIs are known in the art. They are designed to release the API at a predetermined rate in order to maintain a constant drug concentration for a specific period of time. In principle, these dosage forms are characterized by a specific drug release profile, wherein drug release is maintained within a therapeutic window over a prolonged period with the objective of minimizing peak-to-trough fluctuations. This helps to reduce side effects and dosage frequency, thereby improving patient compliance. There is a great variety of sustained release dosage forms. Coated tablets, coated capsules containing pellets, osmotic release oral system (OROS) and other osmotic pressure or matrix systems are only some of a great number of examples of such sustained release systems.

As with all pharmaceutical dosage forms sustained release dosage forms can be categorized by different aspects, e.g. their route of administration, (e.g. oral, inhalational, parenteral, topical administration), their physical appearance (e.g. solid or semi-solid) or their behavior in the human or animal body. While many of these dosage forms are predominantly inert and therefore are not degraded within the human or animal body, others are degraded by enzymes and cells providing the benefit that they do not have to be removed later on by complex surgery. The latter are commonly called biodegradable systems and face a steadily increasing interest in the field of pharmaceutical technology.

One problem of these biodegradable systems is that they require multiple components and/or preparation steps that complicate the formulation process. In particular, various additives may be required in order to provide a composition, which is both suitable for the desired mode of administration as well as qualified to provide the desired release kinetics. For instance, organic solvents are widely used for solubility and homogenization reasons during preparation of these systems. This can lead to major disadvantages in that toxic residues that remain within these systems after preparation can lead to irritations within the foreign body and potential degradation of a protein API.

Additionally, currently available formulations designed to provide extended release of APIs often rely on polymers, which are non-covalently cross-linked by self-organization or gel formation in an aqueous environment. However, these cross-linked matrix systems often lack the desired release kinetics, showing significant initial burst, followed by an exponentially declining release profile. Frequently, this is caused by a rather low polymer segment density and a high fractional water content. The present disclosure addresses these issues and provides related advantages.

Moreover, matrix systems are desired which do not contain APIs and which are suitable for other medical and cosmetic purposes.

In view of the above, there is a need for simpler, safer, and more efficacious biodegradable matrix systems that can be used as drug-delivery compositions having a sustained drug release profile and as matrix material for other medical or cosmetic treatments.

### SUMMARY OF THE INVENTION

As a result of our intensive studies taking the above described problems into consideration, the present inventors were surprised to find that specific hydrophilic matrices including at least one macromolecular non cross-linked compound and having a defined modulus of elasticity as well as a defined moisture content, provide superior sustained release systems that can be used in drug delivery compositions. The hydrophilic matrices, with or without additional drugs, can be used for multiple medical applications, for example as drug delivering implants, or in the cosmetic industry.

According to an embodiment, microparticles are provided which include at least a hydrophilic matrix having a modulus of elasticity of from 0.1 kN/mm² to 10 kN/mm² and a moisture content of from 5% to 60%.

According to an embodiment, a matrix material is provided which includes at least a hydrophilic matrix having a modulus of elasticity of from 0.1 kN/mm² to 10 kN/mm² and a moisture content of from 5% to 60%. According to an embodiment, the matrix material essentially consists of the hydrophilic matrix.

According to an embodiment, a drug-delivery composition is provided, which includes a mixture of at least a hydrophilic matrix having a modulus of elasticity of from 0.1 kN/mm² to 10 kN/mm² and a moisture content of from 5% to 60% and a pharmaceutically active compound.

According to an embodiment that can be combined with any of the other embodiments described herein, a method for manufacturing a drug-delivery composition is provided. The method includes providing a hydrophilic matrix. The hydrophilic matrix has a modulus of elasticity of from 0.1 kN/mm² to 10 kN/mm² and a moisture content of from 5% to 60%. The method further includes providing a pharmaceutically active composition and mixing the hydrophilic matrix and the pharmaceutically active composition to form a drug-delivery composition.

According to an embodiment a method for manufacturing a matrix material includes providing a hydrophilic matrix having a modulus of elasticity of from 0.1 kN/mm² to 10 kN/mm² and a moisture content of from 5% to 60%. The method can further include forming the matrix material into spherical or non-spherical microparticles such as flat particles, or into sheets, and dispersing the microparticles, particles or sheets in solution, ointment, lotion or cream.

According to an embodiment, a method for delivery a drug-delivery composition is provided. The method includes providing a drug-delivery composition having a modulus of elasticity of from 0.1 kN/mm² to 10 kN/mm² and a moisture content of from 5% to 60%, and including a mixture of at least a hydrophilic matrix and a pharmaceutically active composition; and applying the drug-delivery composition into or onto a human or animal body.

Those skilled in the art will recognize additional features and advantages upon reading the following detailed description, and upon viewing the accompanying drawings.

### BRIEF DESCRIPTION OF THE DRAWINGS

The accompanying drawings are included to give a better understanding of the embodiments that are incorporated in and constitute a part of this specification. The drawings illustrate embodiments and together with the description serve to explain principles of embodiments. Other embodiments and many of the intended advantages of embodiments will be readily appreciated, as they become better understood by reference to the following detailed description. The elements of the drawings are not necessarily to scale relative to each other.

FIG. 1 illustrates the compaction of a polymeric matrix by mechanical treatment. Density and/or size of hydrophobic connective spots are increased providing a compact meta-stable matrix.

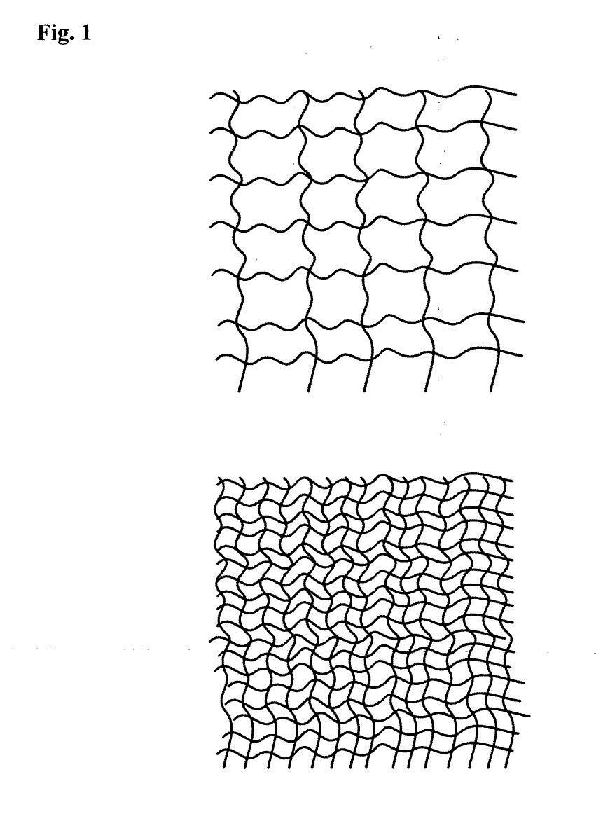

FIG. 2 illustrates a mechanically created compaction of a polymeric matrix by increasing the connective spots of any kind leading to a meta-stable structure of high permanence if the connective spots are of a net attractive type (apolar interaction, H-bridges, polar interactions).

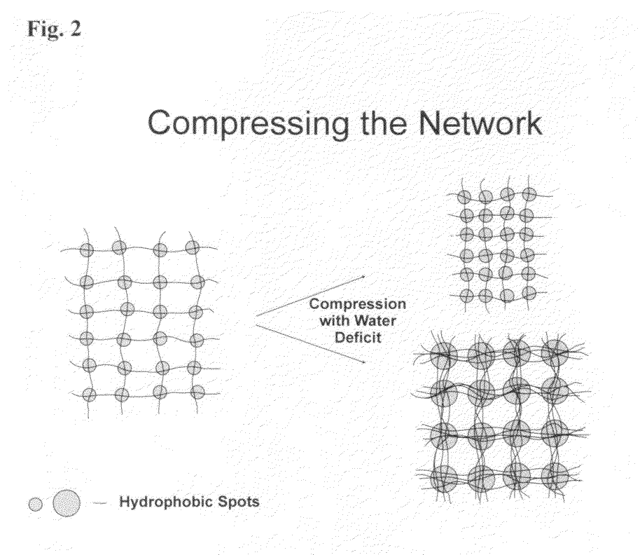

FIG. 3 illustrates a series of compactions compared with aspects of self-organization. The latter one is keeping the segment density more or less constant with growth.

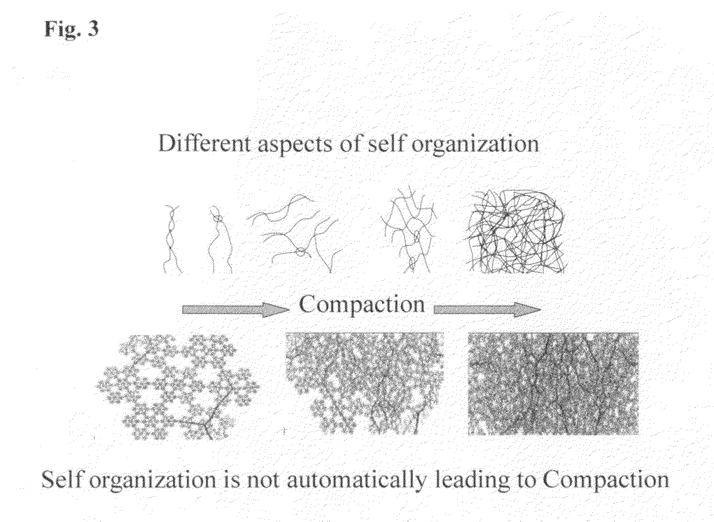

FIG. 4A illustrates a photograph of gelatin B powder used as starting material, while FIG. 4B illustrates a photograph of a macroscopic gelatin matrix of highly compact nature formed by consecutive kneading and wetting processes with a final water content of about 25%. The mass of the dry gelatin powder shown in FIG. 4A is less than finally used.

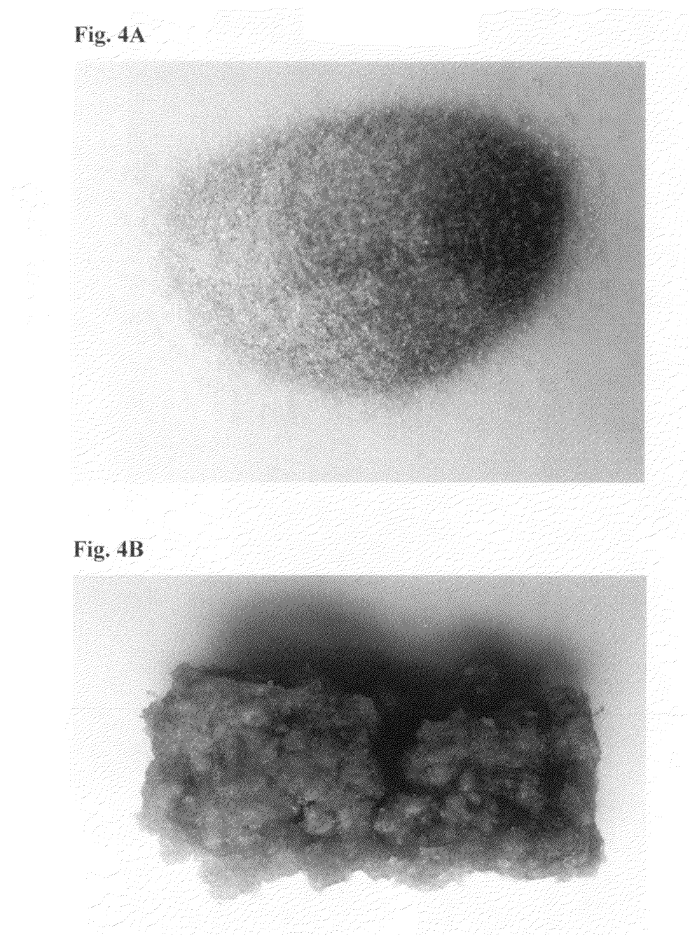

FIG. 5 illustrates antibody release curves from drug-delivery compositions prepared according to several examples illustrating embodiments

### DETAILED DESCRIPTION OF THE INVENTION

Reference will now be made in detail to various aspects of the invention and embodiments. Each aspect is provided by way of explanation and is not meant as a limitation. For example, features illustrated or described as part of one aspect or embodiment can be used on or in conjunction with any other aspect or embodiment to yield yet a further aspect or embodiment. It is intended that the present disclosure includes any such combinations and variations.

In the following, if not otherwise defined, the term “% w/w” refers to the concentration by weight of a component (e.g. macromolecular compound) based on the total weight of the respective entity (e.g. hydrophilic matrix). Furthermore, if not otherwise stated, all measurements were carried out at room temperature.

Moreover, unless otherwise defined, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired and intended properties. At the very least, and not as an attempt to limit the application of the doctrine of equivalents to the scope of the claims, each numerical parameter should at least be construed in light of the number of reported significant digits and by applying ordinary rounding techniques.

For the purpose of this application, the term “naturally occurring” intends to describe materials, e.g. compounds, existing in nature and exist without artificial aid. For instance, naturally occurring proteins are proteins which naturally exist in organisms, e.g. proteins which are encoded in humans without being modified in any contrivable way, e.g. by substituting one or more amino acids.

For the purpose of this application, if not otherwise stated, particle size is determined microscopically or photographically.

Moisture content is determined by formulation and preparation and is determined by a weighing procedure in macroscopic cases.

According to an embodiment, a drug-delivery composition is provided, which includes a mixture of at least a hydrophilic matrix having a modulus of elasticity of from 0.1 kN/mm² to 10 kN/mm² and a moisture content of 5% to 60% and a pharmaceutically active compound.

According to an embodiment, a matrix material includes a hydrophilic matrix having a modulus of elasticity of from 0.1 kN/mm² to 10 kN/mm² and a moisture content of 5% to 60% without a pharmaceutically active compound.

### Drug-Delivery Composition

For the purpose of this application, the term “drug-delivery composition” intends to describe any pharmaceutical dosage form known to those skilled in the art for transporting a pharmaceutically active compound into the human or animal body in order to achieve its desired therapeutic and/or diagnostic effects. Typically, pharmaceutical dosage forms comprise a mixture of a drug components, i.e. pharmaceutically active compound(s), and nondrug components (i.e. excipients). In general, these pharmaceutical dosage forms can be categorized by different aspects, e.g. their route of administration, (e.g. oral, inhalational, parenteral, topical administration) or their physical appearance (e.g. solid, semi-solid, liquid, gaseous). For the purpose of this application, particularly those semi-solid or liquid dosage forms are used, which can be administered topically or via parenteral injection. For instance, such dosage forms comprise ointments, creams, gels, lotions, dispersions, solutions, injection solutions or implants, which is meant to be a non-exhaustive list of possible dosage forms.

According to an embodiment which can be combined with any of the other embodiments described herein, the drug-delivery composition further includes at least one additive. The nature and the type of additive(s) used depend on the dosage form used for administering the drug-delivery composition to a human or animal body. Those skilled in the art know which additives are suitable for each specific dosage form.

According to an embodiment which can be combined with any of the other embodiments described herein, the at least one additive is selected from the group consisting of pharmaceutically accepted fillers, binders, lubricants, coatings or preservatives. These additives can be used alone or in any combination of two or more kinds thereof.

According to an embodiment which can be combined with any of the other embodiments described herein fillers include, but are not limited to lactose, sucrose, trehalose, oligosaccharides glucose, mannitol, sorbitol, calcium carbonate and magnesium stearate, and mixtures thereof.

According to an embodiment which can be combined with any of the other embodiments described herein binders include, but are not limited to saccharides and their derivatives such as disaccharides, e.g. sucrose, lactose; polysaccharides and their derivatives such as starches, cellulose or modified cellulose such as microcrystalline cellulose and cellulose ethers such as hydroxypropyl cellulose (HPC); sugar alcohols such as xylitol, sorbitol or maltitol; or synthetic polymers such as polyvinylpyrrolidone (PVP), and mixtures thereof.

According to an embodiment which can be combined with any of the other embodiments described herein lubricants include, but are not limited to talc or silica, magnesium stearate or stearic acid, and mixtures thereof.

According to an embodiment which can be combined with any of the other embodiments described herein coatings include, but are not limited to synthetic polymers, shellac, corn protein zein, or other polysaccharides derivatives such as cellulose ethers, and mixtures thereof, biopolymers, polyelectrolyte complexes of symmetric or asymmetric type, complexes based on organic and inorganic hybrid electrolytes.

According to an embodiment which can be combined with any of the other embodiments described herein preservatives include, but are not limited to antioxidants like vitamin A, vitamin E, vitamin C, retinyl palmitate, and selenium, amino acids such as cysteine and methionine, citric acid, sodium citrate, synthetic preservatives such as methyl paraben and propyl paraben, and mixtures thereof.

According to an embodiment which can be combined with any of the other embodiments described herein the drug-delivery composition has a moisture content of 5% to 80% particularly of from 8% to 70%, more particularly of 10% to 60%.

According to an embodiment which can be combined with any of the other embodiments described herein the drug-delivery composition has a modulus of elasticity of from 0.01 kN·mm⁻² to 50 kN·mm⁻², more particularly of from 0.1 kN·mm⁻² to 10 kN·mm⁻².

An advantage of drug-delivery compositions according to the present invention is that they show sustained release of the API. For the purpose of this application, the term “sustained release” refers to a drug release profile, wherein drug release is maintained within a therapeutic window over a prolonged period with the objective of minimizing peak-to-trough fluctuations. Particularly, the drug is released from the hydrophilic matrix within a period of few days to several weeks, more particularly from two days to six weeks, typically from 5 days to 3 weeks. This helps to reduce side effects and dosage frequency, thereby improving patient compliance.

According to an embodiment which can be combined with any of the other embodiments described herein the weight ratio between the hydrophilic matrix and pharmaceutically active composition is from 10:1 to 100:1, particularly 10:1 to 50:1, more particularly of from 10:1 to 20:1, typically 15:1. According to an embodiment which can be combined with any of the other embodiments described herein the weight ratio between the hydrophilic matrix and pharmaceutically active composition is from 4:1 to 100:1, particularly 4:1 to 50:1, more particularly of from 4:1 to 20:1.

### Hydrophilic Matrix

For the purpose of this application, the term “hydrophilic matrix” intends to describe a macromolecular polymer system which has polar and/or apolar functional groups. The polymers are not connected to each other by covalent chemical bonds. When being submersed in water the hydrophilic inventive hydrophilic matrix only swells slightly, stays compact and is not gelling the water reservoir. Furthermore, the swelling does not exceed 100% of original body volume per week. There is no chemical modification of pharmaceutical drugs incorporated into the matrix due to the absence of chemical interactions and the presence of physical interactions only. The composition is less sensitive to extremes of heat or pH and no organic solvent are being used.

According to an embodiment which can be combined with any of the other embodiments described herein the hydrophilic matrix has a moisture content of from 5% to 80% particularly of 8% to 70%, more particularly of 10% to 60%.

According to an embodiment which can be combined with any of the other embodiments described herein the hydrophilic matrix has a modulus of elasticity of at least of 0.01 kN·mm², particularly from 0.01 kN·mm⁻² to 50 kN·mm⁻², more particularly of from 0.1 kN·mm⁻² to 10 kN·mm⁻².

According to an embodiment which can be combined with any of the other embodiments described herein, the hydrophilic matrix can be provided as microparticles, sheets or other suitable shapes with or without additional pharmaceutically active compound or compounds.

According to an embodiment which can be combined with any of the other embodiments described herein the hydrophilic matrix is biodegradable.

According to an embodiment which can be combined with any of the other embodiments described herein the hydrophilic matrix is biocompatible.

According to an embodiment which can be combined with any of the other embodiments described herein the hydrophilic matrix includes at least one macromolecular compound.

According to an embodiment which can be combined with any of the other embodiments described herein the macromolecular compound includes at least one polymer having a molecular weight of at least 10,000 Da, particularly of from 10,000 Da to 4 MDa, more particularly of from 20,000 Da to 2 MDa.

According to an embodiment which can be combined with any of the other embodiments described herein the macromolecular compound is selected from gelatin of all modifications (A, B, mixtures, powder, granular). Further suitable compounds are hyaluronic acid, fibrin, polyvinyl alcohol (PVA), polyvinylpyrrolidone (PVP), collagen, alginate, starch, cellulose, chitosan, carboxymethylcellulose, cellulose derivatives, pectin, gum arabic, carrageenan, albumin, fibrinogen, synthetic polyelectrolytes, polyethylenimine, acacia gum, xanthan gum, agar agar, polyvinylalcohol, borax, polyacrylic acids including derivatives, protaminsulfate, casein, and derivatives thereof. According to an embodiment biocompatible, biogenic or synthetic polymers known to the skilled person are suitable as macromolecular compounds. Said biocompatible biogenic or synthetic polymers particularly possess potential physico-chemical interaction opportunities based on the presence of functional groups which could form apolar, polar, salt-bridge or H-bridge connections. According to an embodiment, inorganic polymers such as clay and silica can also be used for the hydrophilic matrix. Furthermore, polyampholytes can be used as polymer components. According to an embodiment, a polymer from the group of biopolymers is used. According to an embodiment, a polymer from the group of polyelectrolyte complex forming substances is used. Such substances typically include two components of opposite charge selected from two polyelectrolytes of opposite charge and a polyelectrolyte and a small ion of opposite charge such as alginate and calcium. According to an embodiment, a polymer from the group of polyampholytes is used. These macromolecular compounds can be used alone or in any combination of two or more kinds thereof.

According to an embodiment which can be combined with any of the other embodiments described herein the macromolecular compound is not cross-linked by covalent bond formation. This means that the macromolecular compound has not actively undergone any step, which would result in covalent cross-linking the polymer chains of the macromolecular compound. The hydrophilic matrix, formed by the macromolecular compound or compounds, is therefore not covalently cross-linked. The hydrophilic matrix can be described as a network of non-covalently cross-linked macromolecular compound or compounds.

According to an embodiment which can be combined with any of the other embodiments described herein the cross-linked macromolecular compound is a naturally occurring compound.

According to an embodiment which can be combined with any of the other embodiments described herein the cross-linked macromolecular compound is a synthetic compound.

According to an embodiment which can be combined with any of the other embodiments described herein the hydrophilic matrix exhibits pores. These pores have an average pore size of from 20 nm to 10 μm, particularly of from 50 nm to 5 μm, and more particularly from 100 nm to 1 μm.

According to an embodiment which can be combined with any of the other embodiments described herein the hydrophilic matrix includes particles.

According to an embodiment which can be combined with any of the other embodiments described herein the particles have an average particle size of from 100 nm to 3 mm, 1 μm to 2 mm, particularly of from 5 μm to 1 mm, and more particularly of from 10 μm to 500 μm.

According to an embodiment which can be combined with any of the other embodiments described herein the particles have an aspect ratio of from 10:1 to 1:1, particularly of from 7:1 to 2:1, more particularly of from 5:1 to 3:1. For the purpose of this application the term “aspect ratio” intends to describe the ratio of the width of the particle to its height.

The above described aspects of the hydrophilic matrix also apply to microparticles and other forms of the hydrophilic matrix that do not include a pharmaceutically active composition so that the microparticles and other forms of the hydrophilic matrix do not form drug-delivery systems. The presence of a pharmaceutically active composition or a pharmaceutically active compound is only optional.

### Pharmaceutically Active Composition

For the purpose of this application, the term “pharmaceutically active composition” intends to describe any pharmaceutical dosage form known to those skilled in the art, which includes or contain a pharmaceutical active compound. For instance, these dosage forms comprise dispersions such as suspensions or emulsions, or solutions.

According to an embodiment which can be combined with any of the other embodiments described herein the pharmaceutically active composition includes a pharmaceutically active compound.

According to an embodiment which can be combined with any of the other embodiments described herein the pharmaceutical active compound is contained in the pharmaceutically active composition in a concentration of from 1 mg/ml to 250 mg/ml, particularly 1 mg/ml to 100 mg/ml, more particularly of from 10 mg/ml to 80 mg/ml, and further particularly of from 15 mg/ml to 30 mg/ml.

According to an embodiment which can be combined with any of the other embodiments described herein the pharmaceutically active composition further includes a liquid component.

According to an embodiment which can be combined with any of the other embodiments described herein the liquid component is selected from hydrophilic solvents, lipophilic solvents and solubilizers, or in any combination of two or more kinds thereof.

According to an embodiment which can be combined with any of the other embodiments described herein the hydrophilic solvent is selected from the group consisting of water, ethanol, glycerol, 1,2-propylene-glycol, low-molecular polyethylene-glycoles (PEG 200, PEG 300, PEG 400), N-methyl-2-pyrrolidone (NMP, Pharmasolve), dimethylacetamide, dimethyl sulfoxide (DMSO), isopropanol, benzyl alcohol and tensides (such as Cremophor EL, Cremophor RH 60, Polysorbat 80 and Solutol HS 15), or mixtures thereof.

According to an embodiment which can be combined with any of the other embodiments described herein the lipophilic solvent is selected from the group consisting of fatty acid esters, isopropylmyristate, -palmitate, -stearate; oleic acid oleyl ester, liquid triglycerides such as Glyceroltriacetat or oils. Said oils are selected from the group consisting of castor oil, clove oil, cassia oil, almond oil, corn oil, arachis oil, cottonseed oil, safflower oil, maize oil, linseed oil, rapeseed oil, soybean oil, caraway oil, rosemary oil, peanut oil, peppermint oil, sunflower oil, eucalyptus oil, olive oil, mentha oil, peppermint oil, eucalyptus oil, bergamot oil, anise oil, fennel oil, or rose oil, or mixtures thereof.

According to an embodiment which can be combined with any of the other embodiments described herein the solubilizer is selected from the group consisting of polyoxyethylene-polyoxypropylene (POE-POP) block copolymers, cyclodextrins (e.g., 3-cyclodextrin, y-cyclodextrin), cyclodextrin derivatives (e.g., sulfobutyl or hydroxypropyl ethers), bile acids, bile acid derivatives, sterol derivatives, alcohols, particularly, fatty alcohols and fatty alcohol derivatives, acids, particularly fatty acids and fatty acid derivatives and tocol derivatives, or mixtures thereof.

According to an embodiment which can be combined with any of the other embodiments described herein, pharmaceutically active composition includes at least one excipient.

According to an embodiment which can be combined with any of the other embodiments described herein, the excipient comprised in the pharmaceutically active composition is selected from the group consisting of monosaccharides, disaccharides, oligosaccharides, polysaccharides like hyaluronic acid, pectin, gum arabic and other gums, albumin, chitosan, collagen, collagen-n-hydroxysuccinimide, fibrin, fibrinogen, gelatin, globulin, polyaminoacids, polyurethane including amino acids, prolamin, protein-based polymers, copolymers and derivatives thereof, and mixtures thereof. An advantage thereof consists in further modifying release characteristics of the drug-delivery composition.

According to an embodiment a drug delivery composition is manufactured, wherein the pharmaceutically active composition includes at least a pharmaceutically active compound without any excipients.

According to an embodiment which can be combined with any of the other embodiments described herein the pharmaceutically active composition is a solution composed of the pharmaceutically active compound as solute, and a liquid component as solvent.

According to an embodiment which can be combined with any of the other embodiments described herein the pharmaceutically active composition is a dispersion composed of the pharmaceutically active compound as dispersed phase, and the liquid component as dispersant.

According to an embodiment which can be combined with any of the other embodiments described herein the dispersed phase is a colloidal dispersed phase.

For the purpose of this application, the term “colloidal dispersed phase” is intended to describe that the dispersed phase has a particle size of from 100 nm to 10 μm, particularly of from 300 nm to 5 μm, more particularly of from 500 nm to 1 μm.

According to an embodiment which can be combined with any of the other embodiments described herein, the dispersion is a gel, a suspension, an emulsion, a lotion, or a cream.

### Pharmaceutically Active Compound

For the purpose of this application, the term “pharmaceutically active compound” intends to describe a pharmaceutical drug which is biologically active and is referred to hereinafter as active pharmaceutical ingredient (API).

According to an embodiment which can be combined with any of the other embodiments described herein, the pharmaceutically active compound is selected from the group consisting of: immunoglobulins, fragments or fractions of immunoglobulins, synthetic substances mimicking immunoglobulins or synthetic, semisynthetic or biosynthetic fragments or fractions thereof, chimeric, humanized or human monoclonal antibodies, Fab fragments, fusion proteins or receptor antagonists (e.g., anti TNF alpha, Interleukin-1, Interleukin-6 etc.), antiangiogenic compounds (e.g., anti-VEGF, anti-PDGF etc.), costimulatory signal inhibitors (e.g. abatacept, alefacept), intracellular signaling inhibitors (e.g JAK1,3 and SYK inhibitors) or other compounds targeting cellular signaling mechanisms or surface antigens on B and T cells (eg anti CD4, 20, 52 etc). Peptides having a molecular mass equal to or higher than 3 kDa, ribonucleic acids (RNA), desoxyribonucleic acids (DNA), plasmids, peptide nucleic acids (PNA), steroids, corticosteroids, an adrenocorticostatic, an antibiotic, an antidepressant or other mood stabilizers, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug, an anticholinergic, an antihistaminic, an antihypertensive, an antihypotensive, an anticoagulant, an antiseptic, an anti-hemorrhagic, an anti-myasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a haemostatic, a hormone and its synthetic or biosynthetic analogue, an immunosuppressant, an immunostimulant, a mitogen, a physiological or pharmacological inhibitor of mitogens, a mineralcorticoid, a muscle relaxant, a narcotic, a neurotransmitter, a precursor of a neurotransmitter, an oligonucleotide, a peptide, a (para)-sympathicomimetic, a (para)-sympatholytic, a protein, a sedating agent, a spasmolytic, a vasoconstrictor, a vasodilatator, a vector, a virus, a virus-like particle, a virustatic, a wound-healing substance, or in any combination of two or more kinds thereof.

### Method for Manufacturing a Drug-Delivery Composition

According to an embodiment which can be combined with any of the other embodiments described herein a method for manufacturing a drug-delivery composition is provided. The method includes providing a hydrophilic matrix. The hydrophilic matrix has a modulus of elasticity of from 0.1 kN/mm² to 10 kN/mm² and a moisture content of from 5% to 60%. The method further includes providing a pharmaceutically active composition and mixing the hydrophilic matrix and the pharmaceutically active composition to form a drug-delivery composition.

According to an embodiment which can be combined with any of the other embodiments described herein a method for manufacturing a matrix material includes providing a powder of a dry hydrophilic material, for example a chemically not cross-linked macromolecular compound or a mixture of chemically not-cross-linked macromolecular compounds, and wetting the dry hydrophilic powder, step-wise or continuously, under continuous or periodic kneading or by alternating steps of wetting and kneading, to obtain a hydrophilic matrix material which has a modulus of elasticity of from 0.1 kN/mm² to 10 kN/mm² and a moisture content of from 5% to 60%.

An advantage of such a manufacturing method consists in achieving a sustained release formulation for pharmaceutically active ingredients with improved release characteristics. In particular, the method allows preparing drug-delivery compositions for sustained release of ingredients characterized by a specific biological activity which otherwise might decrease or even expire.

### Step of Providing a Hydrophilic Matrix

According to an embodiment which can be combined with any of the other embodiments described herein providing a hydrophilic matrix includes, in a first step, providing a macromolecular compound or a mixture of at least two macromolecular compounds as described herein.

According to an embodiment which can be combined with any of the other embodiments described herein providing a macromolecular compound or a mixture of at least two macromolecular compounds includes, in a first step, providing a powder of a macromolecular compound which is not cross-linked (i.e. which is in a non-cross-linked configuration) or of a mixture of at least two macromolecular compounds which are not-cross-linked.

According to an embodiment which can be combined with any of the other embodiments described herein providing a hydrophilic matrix includes, in a second step, adding a solvent to the powder of the macromolecular compound or the mixtures of the at least two macromolecular compounds. Adding the solvent to the powder may be carried out continuously, or, alternatively, in individual, separate steps.

According to an embodiment which can be combined with any of the other embodiments described herein adding the solvent is carried out in a weight ratio powder to solvent from 1:1 to 10:1, particularly from 1:1 to 5:1, more particularly 1:1 to 3:1. Alternatively, the weight ratio powder to solvent can be 2:1 to 1:2.

According to an embodiment which can be combined with any of the other embodiments described herein adding the solvent to the powder is carried out by spraying the solvent onto the powder.

According to an embodiment which can be combined with any of the other embodiments described herein the solvent is a hydrophilic solvent.

According to an embodiment which can be combined with any of the other embodiments described herein the hydrophilic solvent is selected from the group consisting of water, physiological solutions, ethanol, glycerol, 1,2-propylene-glycol, low-molecular polyethylene-glycoles (PEG 200, PEG 300, PEG 400), N-methyl-2-pyrrolidone (NMP, Pharmasolve), dimethylacetamide, dimethyl sulfoxide (DMSO), isopropanol, benzyl alcohol and tensides (such as Cremophor EL, Cremophor RH 60, Polysorbat 80 and Solutol HS 15), or in any combination of two or more kinds thereof.

According to an embodiment which can be combined with any of the other embodiments described herein providing a hydrophilic matrix includes, in a third step, kneading the obtained mixture of powder and solvent to form an elastic body.

According to an embodiment which can be combined with any of the other embodiments described herein kneading the mixture of powder and solvent is carried out in an algorithmic pressing-folding cycle. Said kneading is particularly carried out for 30 seconds to 1 hour, more particularly for 40 seconds to 10 minutes, typically for 50 seconds to 2 minutes.

According to an embodiment which can be combined with any of the other embodiments described herein adding the solvent to the powder and kneading the mixture of powder and solvent is carried out in a continuous manner. This means that small amounts (as related to the powder of hydrophilic matrix) of solvent are continuously added to the powder while continuously kneading the powder/solvent mixture. Particularly, in a first phase, only a part of the solvent to be used is added (particularly sprayed) onto the powder, accompanied by kneading the mixture of powder and solvent, followed by a second phase of adding (particularly spraying) a further part of the solvent onto the powder under continuous kneading the mixture of powder and solvent. According to an embodiment which can be combined with any of the other embodiments described herein this alternation includes from 1 to 50 phases, particularly 1 to 20 phases, more particularly 1 to 10 phases. In this regard, a phase intends to describe a step of spraying a part of the solvent to be used onto the powder under continuous kneading the mixture of powder and solvent.

According to an embodiment which can be combined with any of the other embodiments described herein providing a hydrophilic matrix further includes, in a fourth step, forming the elastic body obtained into a plate or any other regular shape.

According to an embodiment, the solvent can partially evaporate, or driven out of the formed hydrophilic matrix, during kneading to increase the polymer content of the hydrophilic matrix. Driving the solvent, for example an aqueous solution, and/or a liquid component in which the pharmaceutical active compound dissolved or dispersed, out of the hydrophilic matrix or out of the elastic body also can also occur when mixing the hydrophilic matrix and the pharmaceutically active composition. The mechanical treatment forces the solvent and/or liquid component out of the hydrophilic matrix. With increased duration of the mechanical treatment, the portion of the out-driven solvent and/or liquid component increases.

According to an embodiment which can be combined with any of the other embodiments described herein the plate obtained is swelling only slightly, staying compact and is not gelling the water reservoir when being submersed in water. Particularly, the swelling does not exceed 100% of original elastic body volume per week.

According to an embodiment which can be combined with any of the other embodiments described herein the step of providing a hydrophilic matrix includes, in a fifth step, a drying step. This drying step is carried out to obtain a dry, hard and porous body.

### Step of Providing a Pharmaceutically Active Composition

According to an embodiment which can be combined with any of the other embodiments described herein providing a pharmaceutically active composition includes mixing of at least a pharmaceutical active compound (AIP) as described herein, a liquid component as described herein and optionally one or more excipient(s) as described herein. According to an embodiment which can be combined with any of the other embodiments described herein mixing is carried out at a temperature of from 18° C. to 40° C., particularly of from 20° C. to 30° C., typically at 25° C.

### Step of Mixing the Hydrophilic Matrix and the Pharmaceutically Active Composition

According to an embodiment which can be combined with any of the other embodiments described herein the step of mixing the hydrophilic matrix and the pharmaceutically active composition is carried out in a weight ratio of from 1:1 to 10:1, particularly of from 2:1 to 8:1, more particularly of from 3:1 to 6:1, typically of 5:1 to form the drug delivery composition. Furthermore the weight ratio between the hydrophilic matrix and pharmaceutically active composition is from 4:1 to 100:1.

An advantage of this specific weight ratio between the hydrophilic matrix and the pharmaceutically active composition is that the pharmaceutically active composition is completely taken up by the hydrophilic matrix. The swelling of the matrix is limited to the solution containing the pharmaceutical drug which is absorbed thereby efficiently and completely loading the drug into the polymer matrix.

According to an embodiment which can be combined with any of the other embodiments described herein the step of mixing the hydrophilic matrix and the pharmaceutically active composition further includes adding one or more excipient(s) as described herein and one or more solvent(s) as described herein to the mixture.

### Use

According to an embodiment which can be combined with any of the other embodiments described herein the drug-delivery composition or at least one microparticle is used in the treatment of cancer, inflammatory, rheumatic or skin disorders. According to an embodiment which can be combined with any of the other embodiments described herein the drug-delivery composition and/or the microparticle is used for occlusion of blood vessels feeding a tumor downstream. The microparticles can be administered to the blood vessel, for example, by injection, and occlude the blood vessel.

According to an embodiment which can be combined with any of the other embodiments described herein the drug-delivery composition is used for parenteral applications, for filling up cavities or holes of the human body, as artery sealing material after surgery, as implant. For the purpose of this application, the term “parenteral application” intends to describe all dosage forms that are intended to bypass the intestines. Such dosage forms include, but are not limited to injections, infusions, or suppositories.

According to an embodiment which can be combined with any of the other embodiments described herein filling up voids or holes of the human body includes filling up external as well as internal body voids or holes, such as tissue lesions or scars.

For the purpose of this application, the term “artery sealing material” intends to describe all materials, which allow for sealing vascular lesions or injuries due to surgical interventions.

According to an embodiment which can be combined with any of the other embodiments described herein the implants include but are not limited to brain implants, cochlear implants, extra ocular implants, retinal implants, dental implants, breast implants, contraceptive implants, prosthetic implants, subdermal implants or transdermal implants.

According to an embodiment which can be combined with any of the other embodiments described herein the drug-delivery composition is used for topical, cosmetic applications, masks, or coverings of human body parts.

According to an embodiment which can be combined with any of the other embodiments described herein topical, cosmetic applications are selected from the group consisting of semisolid or liquid compositions such as ointments, creams, emulsions, suspensions, gels or solutions.

According to an embodiment which can be combined with any of the other embodiments described herein including masks as plane matrixes coating parts of the body surface in a conformal manner, covering large areas of the body surface and masks fitting to each other geometrically in any area.

According to an embodiment which can be combined with any of the other embodiments described herein covering human body parts including but not limited to dressing materials, e.g. dressing materials for covering wounds, patches, or inlets of band-aids.

According to an embodiment which can be combined with any of the other embodiments described herein the drug-delivery composition is used in the field of tissue engineering, e.g. as biocompatible scaffold for in vitro and in vivo culturing of cells.

### Method for Delivery

According to an embodiment, a method for delivery a drug-delivery composition is provided. The method includes providing a drug-delivery composition having a modulus of elasticity of from 0.1 kN/mm² to 10 kN/mm² and a moisture content of from 5% to 60%, and including a mixture of at least a hydrophilic matrix and a pharmaceutically active composition; and applying the drug-delivery composition into a human or animal body.

According to an embodiment which can be combined with any of the other embodiments described herein the step of applying the drug-delivery composition into a human or animal body includes injecting the composition into a human or animal body.

According to an embodiment which can be combined with any of the other embodiments described herein injecting means intraocular injecting, subcutaneous injecting, intramuscular injecting, intraperitoneal injecting, intravenous injecting, direct injection into tissue or cavities by means of catheter or other direct device technology.

According to an embodiment, a method for manufacturing a polymer body includes providing a dry polymer powder which includes a polymer; and kneading the dry polymer powder under consecutive addition of small amounts of an aqueous solution to form an elastic polymer body having a polymer-water weight ratio between 2:1 to 1:2.

According to an embodiment, a method for manufacturing a drug delivery composition includes: providing a dry polymer powder comprising a polymer; kneading the dry polymer powder under consecutive addition of given amounts of an aqueous solution to form a hydrophilic polymer matrix which forms an elastic polymer body, wherein the hydrophilic polymer matrix of the elastic polymer body has a polymer-water weight ratio between 2:1 to 1:2; providing a drug powder comprising a pharmaceutically-active macromolecular drug selected from the group consisting of a bioactive protein and a nucleic acid; adding, at ambient temperature or at a temperature below ambient but above the water-freezing point, the drug powder to the elastic polymer body formed by the hydrophilic polymer matrix and kneading the drug powder and the elastic polymer body to form a semi-solid or elastic drug delivery composition, wherein the drug delivery composition is composed of at least 80, and typically of at least 90 wt % of the hydrophilic polymer matrix.

According to an embodiment, a method for manufacturing a drug delivery composition includes: providing a powder of a pharmaceutically-active macromolecular drug selected from the group consisting of a bioactive protein and a nucleic acid; mixing the powder at least with water to obtain an aqueous drug suspension; providing a dry polymer powder comprising a polymer; kneading, at a temperature below ambient but above the water-freezing point, the polymer powder under consecutive addition of given amounts of the aqueous drug suspension to form a semi-solid or elastic drug delivery composition having a polymer-water weight ratio between 2:1 to 1:2, wherein the drug delivery composition is composed of at least 80, and typically of at least 90 wt % of the polymer and the pharmaceutically-active macromolecular drug.

According to an embodiment, a sustained drug-releasing dosage form includes a drug delivery composition. The drug delivery composition includes a matrix having a polymer, a pharmaceutically-active macromolecular drug selected from the group consisting of a bioactive protein and a nucleic acid, wherein the pharmaceutically-active macromolecular drug is homogeneously distributed throughout the matrix, and wherein the drug delivery composition is paste-like, semi-solid or elastic and composed of at least 80, and typically of at least 90 wt % of the polymer. The drug delivery composition is capable-of-sustained release of the pharmaceutically-active macromolecular drug over a sufficiently long period so that at least 10%, preferably at least 20% and more preferably at least 30% of the pharmaceutically-active macromolecular drug is released after 2 weeks, wherein the dosage form has a size and shape suitable for injection into a human or mammalian eye.

The present invention shall be described in more detail in the following Examples.

### Example 1

To 5 g of dry gelatin powder, 3 g of water is gradually added under a permanent mechanical kneading process (by a couple of tiny sprays from a water reservoir). Through ongoing kneading the wet mass is transformed into an elastic solid in a rather short period of time (about 1 minute). For example, if the elastic body has been formed to a plate and is loaded by a weight of 5 kp the height is changed by only a few percent of its original, indicating that a highly elastic gelatin body has been obtained.

If this elastic gelatin body is submersed in water it swells only slightly and stays compact and is not gelling the water reservoir. The swelling is not exceeding 100% of original gelatin body volume per week.

### Example 2

The wet solid elastic gelatin body prepared according to Example 1 is air dried (could be done in a digestorium or under elevated temperature or by lyopholization) and after forming a dry and hard porous body it is milled into particulate form (macro- or microparticles). These microparticles remain as microparticles for a few days, however, possess a lower elasticity when compared to the original elastic gelatin body

### Example 3

The dry body obtained according to Example 2 is re-wetted by any solvent or solvent mixture wetting the material and not destroying the structure, e.g. plant oil, ethanol, pharmaceutically accepted solvents, liquid carbon hydrates or tocopherol, or any other liquid organic substance.

### Example 4

Dry gelatin (10 g) is mixed with small aliquots (1 g) of water in a series of consecutive steps under steady kneading up to a gelatin-to-water ratio of 2. Continuous kneading/mixing for 3 minutes leads to a single gelatin body of well-defined elasticity but only small plasticity. The introduction of this gelatin body into water at room temperature results in a stable, gelatinous body, which does not swell significantly over a period of days and weeks.

### Example 5

Dry gelatin (10 g) is mixed with 5 g of water. In contrast to the preparation process of example 4, the mechanical kneading was carried out for a time period of 10 seconds only. The total disintegration of the gelatin body is observable about ten hours after formulation.

### Example 6

5 ml of water was added to a mixture of 5 g of carboxymethylcellulose and 5 g of chitosan. This mixture was mechanically kneaded for 3 minutes and a solid elastic body was formed and suspended in water at room temperature. This same body was observed after predefined periods of time. A continuous swelling process is observed during a first period of 42 hours, however, not leading to disintegration of the solid mass. In comparison to this approach, disintegration was observed when the same composition was kneaded for only 10 seconds as the gelatin system of Example 5.

Further observation of the mechanically kneaded composition up to 145 hours after preparation demonstrates an increasing tendency of disintegration. The stabilization effect via the mechanical treatment is clearly visible during the first 42 hours; however, it is much less expressed as compared to the gelatin composition of example 1.

### Example 7

Equal amounts of dry carboxymethylcellulose and dry chitosan (5 g each) are mixed with 5 g of acetic acid (pH 3) and a small amount (less than 1 g) of plant oil. The mixture is mechanically treated, i.e. kneaded, for 3 minutes and formed into a spherical body which is suspended into water at room temperature and observed over time. Despite a clearly visible swelling there is no disintegration during the 27 hours observation period. The CMC/chitosan system is much less stable than the gelatin system (example 4). If the system is mechanically treated for only 10 seconds the disintegration of the spherical body after suspension into water at room temperature is starting more or less directly (not shown) and its behavior is, at least in principle, comparable to the gelatin system of example 5. The gelatin system shows a little more stability.

### Example 8

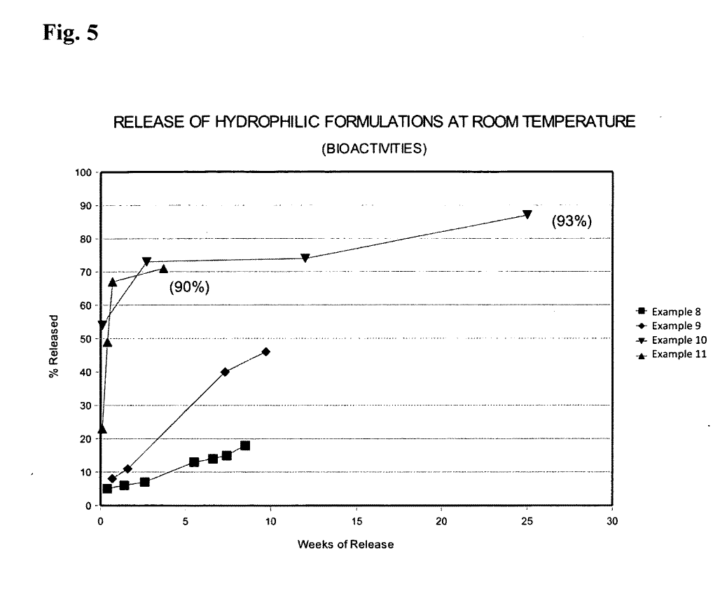

First, a calcium alginate film was prepared by addition of a calcium chloride solution to 1.0 g aqueous alginate gel (2%, 0.01% sodium azide) in a flat bowl. After 10 minutes the resulting film was separated from mold and dried for 2 minutes on white filter paper. Second, 2 mg of antibody 1 of the type of gamma globulin was placed onto the center of the film. Third, the film was folded together and kneaded by hand for 7 minutes forming ultimately a spherical particle. To this particle, 1.0 g of an isotonic sodium chloride solution was added. The release of antibody 1 was determined spectroscopically by the UV 280 nm method under sink conditions (cp. FIG. 5, Example 8). Ultimately we observed a very slow release rate (18.5% after 8.5 weeks).

### Example 9

First, a calcium alginate film was prepared by addition of a calcium chloride solution to 1.0 g aqueous alginate gel (2%, 0.01% sodium azide) in a flat bowl. After 10 minutes the resulting film was separated from the mold and dried for 2 minutes on white filter paper. Second, 25 mg of micro-crystalline cellulose and 50 mg of an aqueous antibody 2 (of the gamma globulin type) solution was placed onto the center of the film. Third, the film was folded together and kneaded by hand for 7 minutes forming ultimately a spherical particle. To this particle 1.2 g of an isotonic sodium chloride solution was added. The release of antibody 2 was determined spectroscopically by the UV 280 nm method under sink conditions (cp. FIG. 5, Example 9). Ultimately we observed a medium release rate of 46% in 9.7 weeks. After 3.7 weeks about 90% of released antibody 2 is active.

### Example 10

66 mg of an antibody 2 solution (25 mg/ml) was added to 24 mg of micro-crystalline cellulose and 90 mg of castor oil. This mixture was mechanically treated using a glass rod for 1 minute. The resulting product was mixed with 1.5 g of an aqueous alginate gel (2%, 0.01% sodium azide) and then dropped into a cold aqueous calcium chloride solution (18%) under stirring (magnetic stirrer 500 U/min). The obtained capsules were separated from suspension and washed two times with double distilled water. The resulting alginate capsules were added to 3.0 g of an isotonic sodium chloride solution (0.01% azide). The release of antibody 2 was determined spectroscopically by the UV 280 nm method under no-sink conditions (cp. FIG. 5, Example 10). This system represents a mixed hydrophilic/hydrophobic system. The resulting release behavior is demonstrating a two-phase characteristic; after a fast release period of 73% in 2.7 weeks there is a slowing down to another 14% over the next 22 weeks. After 25 weeks of release about 93% or the released antibody 2 is bio-active as checked by ELISA.

### Example 11

200 mg of an antibody 3 (of gamma globulin type) solution (50 mg/ml) was added to 80 mg micro-crystalline cellulose and 90 mg of castor oil. This mixture was mechanically treated using a glass rod for 1 minute. The resulting product was mixed with 1.0 g of an aqueous alginate gel (2%) and then dropped into a cold aqueous calcium chloride solution (18%) under stirring (magnetic stirrer 500 U/min). The obtained capsules were separated from suspension and washed two times with double distilled water and finally added to 5.0 g of an isotonic sodium chloride solution. The release of antibody 3 was determined spectroscopically by the UV 280 nm method under no-sink conditions (cp. FIG. 5, Example 11). Ultimately, we observed a similar behavior as in previous EXAMPLE 7. After about 4 weeks of release about 90% of the released antibody 3 is bio-active as determined by ELISA.

1-51. (canceled)

52. A drug-delivery composition, comprising:
at least a hydrophilic matrix, wherein said hydrophilic matrix has a modulus of elasticity of from about 0.1 kN/nm² to 10 kN/nm² and a moisture content of from about 5% to about 60% or from about 10% to about 60%; and
a pharmaceutically active compound.

53. The drug-delivery composition of claim 52, wherein the weight ratio between the hydrophilic matrix and the pharmaceutically active compound is from 4:1 to 100:1.

54. The drug-delivery composition of claim 52, wherein the hydrophilic matrix is biodegradable and biocompatible.

55. The drug-delivery composition of claim 52, wherein the hydrophilic matrix comprises at least one macromolecular compound, said macromolecular compound comprising at least one polymer having a molecular weight of at least 10,000 Da, particularly from about 10,000 Da to 4 MDa, more particularly from about 20,000 Da to 2 MDa.

56. The drug-delivery composition of claim 55, wherein the macromolecular compound is selected from the group consisting of gelatin, hyaluronic acid, fibrin, collagen, or alginate.

57. The drug-delivery composition of claim 55, wherein the macromolecular compound is not cross-linked.

58. The drug-delivery composition of claim 52, wherein the hydrophilic matrix comprises particles having an average particle size of from about 100 nm to about 3 mm.

59. The drug-delivery composition of claim 58, wherein the particles have an aspect ratio of from about 10:1 to 1:1.

60. A method for manufacturing the drug-delivery composition of claim 52, said method comprising:
providing a hydrophilic matrix, wherein providing said hydrophilic matrix comprises providing at least one macromolecular compound that is not cross-linked in a powder form, adding a solvent to said powder, kneading said powder and said solvent so that the hydrophilic matrix is in an elastic form, and forming said hydrophilic matrix in elastic form into a plate form; and
mixing said hydrophilic matrix in a plate form with a pharmaceutically active compound.

61. The method of claim 60, wherein adding the solvent to the powder is carried out in a weight ratio of powder to solvent from about 1:1 to 10:1.

62. The method of claim 60, wherein the solvent is added to the powder by spraying the solvent onto the powder.

63. The method of claim 60, wherein the solvent is selected from the group consisting of water, physiological solutions, ethanol, glycerol, 1,2-propylene-glycol, low molecular polyethylene glycol, or a combination thereof.

64. The method of claim 60, wherein kneading the mixture of powder and the solvent is carried out in an algorithmic pressing folding cycle and is carried out for 50 seconds to 2 minutes.

65. The method of claim 60, wherein the adding of solvent to the powder and the kneading of the mixture and solvent is carried out in a continuous manner.

66. The method of claim 60, wherein in a first phase, only a part of the solvent to be used is sprayed onto the powder, accompanied by kneading the mixture of powder and the solvent, followed by a second phase of spraying a further part of the solvent onto the powder under continuous kneading the mixture of the powder and the solvent.

67. The method of claim 66, wherein said method comprises carrying out 1 to 50 phases.

68. The method of claim 60, wherein swelling of the plate does not exceed 100% of original elastic body volume per week when being submerged in water.

69. The method of claim 60, further comprising:
drying the hydrophilic matrix.

70. A method of treating a human or an animal with a pathophysiological condition, comprising:
administering the drug delivery composition of claim 52 to the human or animal.

71. The method of claim 70, wherein said drug delivery composition is administered by a means selected from the group consisting of parental administration, implantation, intraocular injection, subcutaneous injection, intramuscular injection, intraperitoneal injection, inhalation, intranasal administration, topical administration, administration by catheter or similar drug delivery devices, administration directly into cavities or holes of the individual's body, administration as a mask, administration to cover human body parts, administration to feed a downstream tumor, and administration into a blood vessel that feeds to a tumor to cause occlusion of the blood vessel.

72. The method of claim 70, wherein said pathophysiological condition is selected from the group consisting of cancer, inflammatory disease, rheumatic disease, skin disease, and as post-surgical intervention for sealing an artery.

73. A solid elastic material, comprising:
a hydrophilic matrix having a modulus of elasticity of from about 0.1 kN/mm² to about 10 kN/mm² and a moisture content of from about 5% to 60%.

74. The solid elastic material of claim 73, wherein the hydrophilic matrix further comprises:
a pharmaceutically active compound.

75. The solid elastic material of claim 73, wherein the material is at least of one shape and cut.

76. The solid elastic material of claim 73, wherein the material is in at least one of wet or non-dried form.

77. The solid elastic material of claim 73, wherein said material is used in ways selected from the group consisting of topical cosmetic application, mask, covering of human body part, parental administration as drug delivery material, scaffold for filling up a cavity or a hole in human or animal body, implant, sealing body after surgery or catheterization, as inlet of band-aid and for covering of wounds.

78. A microparticle, comprising the solid elastic material of claim 73, wherein said microparticle is causes occlusion of blood vessels, feeds a downstream tumor or a combination thereof.

79. The microparticle of claim 78, wherein the hydrophilic matrix comprises at least one not chemically cross-linked macromolecular compound comprising at least one polymer having a molecular weight of from about 20,000 Da to 2 MDa.

80. The microparticle of claim 79, wherein the macromolecular compound is selected from the group consisting of gelatin, hyaluronic acid, fibrin, collagen, or alginate.

81. A method of treating cancer, comprising:
administering the microparticle of claim 79 into a blood vessel that feeds into a tumor, wherein said administration of the microparticle causes occlusion of the blood vessel.

82. A method of manufacturing a polymer body, comprising:
providing a polymer in a dry powder form; and
kneading the dry powder under consecutive addition of small amounts of an aqueous solution to form an elastic polymer body having a polymer-water weight ratio from about 2:1 to about 1:2.

83. A method for manufacturing a drug delivery composition, comprising:
providing a polymer in a dry powder form;
kneading the dry powder under consecutive addition of given amounts of an aqueous solution to form a hydrophilic polymer matrix, wherein said hydrophilic polymer matrix forms an elastic polymer body, wherein the hydrophilic polymer matrix of the elastic polymer body has a polymer-water weight ratio from about 2:1 to about 1:2;
providing a drug powder comprising a pharmaceutically-active macromolecular drug selected from the group consisting of a bioactive protein and a nucleic acid;
adding the drug powder to the elastic polymer body formed by the hydrophilic polymer matrix at ambient temperature or at a temperature below the ambient temperature and above the water-freezing point; and
kneading the drug powder and the elastic polymer body to form a semi-solid or elastic drug delivery composition, wherein the drug delivery composition is composed of at least about 80 and typically at least about 90 wt % of the hydrophilic polymer matrix.

84. A method for manufacturing a drug delivery composition, comprising:
providing a pharmaceutically active macromolecular drug in a powder form, wherein said drug is selected from the group consisting of a bioactive protein and a nucleic acid;
mixing the powder at least with water to obtain an aqueous drug suspension;
providing a polymer in a dry powder form; and
kneading the dry powder under consecutive addition of given amounts of the aqueous drug suspension at a temperature below ambient but above the water-freezing point to form a semi-solid or elastic drug delivery composition having a polymer-water weight ratio between about 2:1 to about 1:2, wherein the drug delivery composition is composed of at least about 80 and typically at least about 90 wt % of the hydrophilic polymer matrix.

85. A sustained drug-releasing dosage form, comprising:
a drug delivery composition, comprising:
a matrix comprising a polymer;
a pharmaceutically-active macromolecular drug, wherein said drug is selected from the group consisting of a bioactive protein and a nucleic acid, wherein the pharmaceutically-active macromolecular drug is homogenously distributed throughout the matrix;
the drug delivery composition being paste-like, semi-solid, or elastic, wherein said drug delivery composition is composed of at least about 80, and typically of at least about 90 wt % of the polymer;
wherein the drug delivery composition is capable of sustained release of the pharmaceutically-active macromolecular drug over a sufficiently long period so that at least about 10%, preferably at least about 20% and more preferably, at least about 30% of the pharmaceutically active macromolecular drug has been released after 2 weeks, wherein the dosage form has a size and shape suitable for injection into a human or a mammalian eye.
